(19) United States
(12) Patent Application Publication
(10) Pub. No.: US 2020/0368336 A1
(43) Pub. Date: Nov. 26, 2020

(54) METHOD FOR PREPARING PERSONALIZED CANCER VACCINE

(71) Applicant: SHANGHAI JENOMED BIOTECH CO., LTD., Shanghai (CN)

(72) Inventors: Yuejin HUANG, Shanghai (CN); Pan YANG, Shanghai (CN)

(21) Appl. No.: 16/768,768

(22) PCT Filed: Dec. 3, 2018
(86) PCT No.: PCT/CN2018/118984

§ 371 Date: Jun. 9, 2020

(30) Foreign Application Priority Data
Dec. 1, 2017 (CN) 201711252479.8

(51) Int. Cl. A61K 39/00; C12Q 1/6886; G16B 30/10; C12N 5/0784; C12N 5/0783

(52) U.S. Cl. CPC A61K 39/0011; C12Q 1/6886; G16B 30/10; C12N 2502/1114; C12N 5/0638; C12N 2501/999; C12N 5/0639

(57) ABSTRACT

A method for preparing a personalized cancer vaccine is disclosed. CTC as well as DNA and RNA or ctDNA and ctRNA are separated or enriched to a certain ratio; 13-20 types of DNAs having tumor-specific somatic mutations, RNA, or short-chain peptide, i.e., tumor neoantigen, which can cause a change in a protein sequence and can be closely bind to an human HLA type I or II receptory and TCR, and can further activate CD8+T cells or CD4+T helper cells, are separated. Further, a personalized cancer vaccine is prepared within 4-6 weeks, and is used for stimulating immune response in a cancerous object. Furthermore, antigens capable of stimulating anti-cancer immunity can be captured under an almost noninvasive condition, so that sequencing time and introduced errors can be reduced.

### TECHNICAL FIELD

The present invention belongs to the field of biomedical technology, in particular to a preparation method of personalized cancer vaccine, in particular to a method for collecting and screening antigen fragments containing tumor-specific somatic mutations from body fluids of cancer-bearing subjects, thereby preparing personalized cancer vaccines.

### BACKGROUND

Cancer occurs when certain cells in the patient's body have gene mutations, uncontrolled proliferation and differentiation, and eventually develop into malignant tumors. There are many neoantigen proteins encoded by mutant genes on the surface of cancer cells. Under normal circumstances, they should be recognized by the human immune system in time and trigger an immune response to clear these cancer cells. However, under pathological conditions, tumor cells develop and differentiate rapidly, and new mutations constantly occur, making the body's immune system unable to recognize in time. Coupled with the immunosuppression formed in the tumor microenvironment, the immune system may be completely incapable of responding. Although currently more advanced immunotherapy treatments, such as CAR-T technology, can transform T cells in vitro, enhance their tumor cell immune recognition and response capabilities, and inject them back into patients after in vitro amplification, but after injection, the patient can't replicate these cells. Of course, some of the immune cells imported into the body may be latent for a long time and become “memory cells”, so that they may “recover” in the future. But these cells have been genetically modified, what are the problems caused by lurking in the human body for a long time? There is no answer in the short term. At the same time, excessively lowering the immune response threshold may lead to excessive immune response and various inflammations. The most advanced personalized CAR-T technology is currently only effective for some patients with individual cancers, and recently there have been deaths of patients caused by allogeneic CAR-T drugs.

Cancer immunotherapy needs a different approach. The reason why neoantigen proteins encoded by the mutated genes and presented on the surface of cancer cells cannot cause an immune response may be that the expression of these abnormal proteins is not high enough to trigger immune recognition and immune response. The development of tumor genome sequencing and the progress of cancer immunotherapy have made it possible to use these abnormal tumor neoantigen proteins to make cancer vaccines (Ott P A Nat 2017; 547: 217-221, Epub 2017 Jul. 5; Sahin U et al. Nat 2017; 547: 222-226, Epub 2017 Jul. 5). The so-called personalized cancer vaccine, that is, an anti-cancer vaccine customized according to the mutations related to the respective tumor cells of the cancer subject, is an advanced stage of the development of personalized medicine (precision medicine). However, how to efficiently obtain key antigens from tissues and safely apply them to the required objects to effectively inhibit tumors still faces many challenges of cancer vaccines. For example, the preparation time of the vaccine is longer, which takes 6-8 weeks; samples must be obtained by surgical removal of cancerous tissue in advanced patients in order to detect and confirm tumor somatic mutations. Both the long cycle of cancer vaccines and the invasive access to it are difficult to meet the huge clinical treatment needs of cancer patients.

### SUMMARY OF THE INVENTION

In a first aspect of the present invention, it provides a method for preparing a personalized cancer vaccine, comprising the following steps:

(a) providing a first sample sequencing data set A1 and a first control sequencing data set R1 corresponding to a subject; and/or providing a second sample sequencing data set A2 and a second control sequencing data set R2 corresponding to a subject,

wherein the first sample data set A1 and the first control sequencing data set R1 are obtained by a method including the following steps:

t1) providing a first sample, and the first sample is a sample containing a CTC cell and a normal body fluid cell;

t2) performing CTC cell enrichment treatment on the first sample, thereby obtaining an enriched first sample, wherein in the enriched first sample, the CTC cell abundance C1≥5% and the normal body fluid cell abundance C2≤95%, based on the total number of all cells in the enriched sample, and the ratio of the CTC cell abundance C1 to the normal body fluid cell abundance C2 is recorded as B1 (i.e., B1=C1/C2);

t3) extracting DNA and/or RNA from the enriched first sample, thereby obtaining a first nucleic acid sample, wherein the first nucleic acid sample includes a nucleic acid sample from a CTC cell and a nucleic acid from a normal body fluid cell; and

t4) sequencing the first nucleic acid sample, wherein the nucleic acid sample from a normal body fluid cell in the first nucleic acid sample is used as a control for the nucleic acid sample from a CTC cell, thereby obtaining the first sample sequencing data set A1 and the first control sequencing data set R1, wherein the first sample sequencing data set A1 corresponds to the sequencing data set of a CTC cell, and the first control sequencing data set R1 corresponds to the sequencing data set of a normal body fluid cell;

wherein the second sample data set A2 and the second control sequencing data set R2 are obtained by a method including the following steps:

w1) providing a second sample, and the second sample is a sample containing a circulating tumor DNA (ctDNA) and a circulating tumor RNA (ctRNA) and other free DNA (cfDNA) and free RNA (cfRNA);

w2). enriching the second sample to obtain an enriched second nucleic acid sample;

wherein, the enriched second nucleic acid sample includes ctDNA and ctRNA from a CTC cell and cfDNA and cfRNA from a normal body fluid cell, wherein based on the total weight of all nucleic acids, the content of ctDNA and ctRNA L1≥5%, while the content of cfDNA and cfRNA from a normal cell L2≤95%, and the ratio of the content L1 to L2 is recorded as B2 (i.e., B2=L1/L2);

w3). sequencing the second nucleic acid sample, wherein the cfDNA and cfRNA from a normal cell in the second nucleic acid sample are used as a control for ctDNA and ctRNA from a CTC cell to obtain a second sample sequencing data set A2 and a second control sequencing data set R2, wherein the second sample sequencing data set A2 corresponds to the sequencing data set of a CTC cell, and the second control sequencing data set R2 corresponds to the sequencing data set of a normal body fluid cell;

(b). performing sequence alignment treatment on the first sample sequencing data set A1 and the first control sequencing data set R1, or the second sample sequencing data set A2 and the second control sequencing data set R2, respectively, thereby obtaining a first candidate data set S1 or a second candidate data set S2; wherein any sequence element in the first candidate data set S1 is an element present in the A1 but not present in the R1; and any sequence element in the second candidate data set S2 is an element present in the A2 but not present in the R2;

(c). performing an HLA type I or II receptor affinity prediction analysis on any sequence element in the first candidate data set S1 and/or the second candidate data set S2 to obtain a primarily selected sequence element, the primarily selected sequence element is a sequence element that binds tightly to the HLA type I or II receptor (IC50≤500 nm, preferably, 100 nm);

(d). based on the primarily selected sequence element, synthesizing a DNA, RNA, and short peptide chain corresponding to the primarily selected sequence element;

(e). using the synthesized DNA, RNA, and short peptide chain to perform an in vitro T-cell receptor (TCR) binding test and CD8 + T cell and/or CD4 + T helper cell activation test to obtain 10-30 secondarily selected sequence elements, wherein the secondly selected sequence elements can bind to TCR and activate CD8 + T cells and/or CD4 + T helper cells;

(f) based on the secondarily selected sequence elements, synthesizing DNA, RNA and peptide chains corresponding to the secondarily selected sequence elements;

(g). mixing the DNA, RNA, and peptide chains synthesized in the previous step with a pharmaceutically acceptable carrier to prepare a pharmaceutical composition, which is a personalized cancer vaccine.

In another preferred embodiment, in the enriched first sample, the CTC cell abundance is 5% to 95% (preferably 10-90%) and the normal body fluid cell abundance is 95% to 5% (preferably 90-10%), and the CTC cell abundance and the normal body fluid cell abundance are added up to 100%.

In another preferred embodiment, in the enriched second sample, the content of ctDNA and ctRNA from a CTC cell is 5% to 95% (preferably 10-90%) and the content of cfDNA and cfRNA from a normal cell is 95% to 5% (preferably 90-10%), and the content of ctDNA and ctRNA of a CTC cell and the content of cfDNA and cfRNA of a normal cell are added up to 100%.

In another preferred embodiment, in the first nucleic acid sample, the weight ratio B2 of the nucleic acid sample from a CTC cell to the nucleic acid sample from a normal body fluid cell is equal to or substantially equal to B1.

In another preferred embodiment, the first control sequencing data set R1 corresponds to the sequencing data set of a normal PBMC cell.

In another preferred embodiment, the second control sequencing data set R2 corresponds to the sequencing data set of a normal PBMC cell.

In another preferred embodiment, in step (t4), “using the nucleic acid sample from a normal body fluid cell as a control for the nucleic acid sample from a CTC cell” refers to the sequencing data is subjected to classification and/or analysis with reference to the ratio B1 of CTC cell abundance C1 and normal body fluid cell abundance C2.

In another preferred embodiment, in step (w3), “using cfDNA and cfRNA from a normal cell as a control of ctDNA and ctRNA from a CTC cell” refers to the sequencing data is subjected to classification and/or analysis with reference to the ratio B2 of the ctDNA and ctRNA content L1 of a CTC cell to the cfDNA and cfRNA content L2 of a normal cell.

In another preferred embodiment, in the classification and/or analysis, for the two types of sequencing data D1 and D2 at the same location or position, if the following Formula Q1 is met, the sequencing data D1 is classified as CTC sequencing data, and the sequencing data D2 is classified as sequencing data of a normal body fluid cell

RD1/(RD1+RD2)≈C1/(C1+C2) (Q1)

wherein,

RD1 is the frequency of occurrence (or abundance, such as read depth) of sequencing data D1 (such as read or a related sequence thereof)

RD2 is the frequency of occurrence (or abundance, such as read depth) of sequencing data D2 (such as read or a related sequence thereof)

C1 is the abundance of a CTC cell in the enriched first sample;

C2 is the abundance of a normal body fluid cell in the enriched first sample.

In another preferred embodiment, in the classification and/or analysis, for the two types of sequencing data E1 and E2 at the same location or position, if the following Formula Q2 is met, the sequencing data E1 is classified as ctDNA and ctRNA sequencing data of a CTC cell, and the sequencing data E2 is classified as ctDNA and ctRNA sequencing data of a normal cell

RE1/(RE1+RE2)≈L1/(L1+L2) (Q2)

wherein

RE1 is the frequency of occurrence (or abundance, such as read depth) of sequencing data E1 (such as read or a related sequence thereof)

RE2 is the frequency of occurrence (or abundance, such as read depth) of sequencing data

E2 (such as read or a related sequence thereof)

L1 is the content of ctDNA and ctRNA of a CTC cell in the enriched second sample;

L2 is the content of ctDNA and ctRNA of a normal cell in the enriched second sample.

In another preferred embodiment, in step (w2), the enriching includes performed by one or more methods selected from the group consisting of: capturing based on cell size (filtration method) or positive capturing based on tumor surface markers (immunological method).

In another preferred embodiment, in step (t2), the enriching includes performed by one or more methods selected from the group consisting of molecular sieve, methylation separation, filtration centrifugation, and a combination thereof.

In another preferred embodiment, the sequencing includes performed by one or more methods selected from the group consisting of: preliminary screening Ultra low pass-WGS, WES, or RNA-seq.

In another preferred embodiment, the sequence element is the following group: a DNA sequence element, RNA sequence element, and/or peptide chain sequence element.

In another preferred embodiment, the DNA sequence element contains 2-5 DNA variants, and each DNA variant contains at least 5 short peptide chain coding sequences; and/or

the RNA sequence element contains 2-5 RNA variants, and each RNA variant contains at least 5 short peptide chain coding sequences; and/or

the peptide chain sequence element contains 5-100 amino acids.

In another preferred embodiment, the peptide chain sequence element is preferably 10-80 amino acids, more preferably 15-50, such as 20, 30 or 40 amino acids.

In another preferred embodiment, the “sequence element binding to HLA type I or II receptor” refers to the peptide sequence corresponding to the sequence element (i.e., the peptide chain sequence element itself, or the peptide sequence encoded by the RNA sequence element/DNA sequence element) is capable of binding to HLA type I or II receptor.

In another preferred embodiment, the normal body fluid cell includes leukocyte, monocyte, lymphocyte and the like.

In another preferred embodiment, the method is also used for early diagnosis of cancer.

In another preferred embodiment, the method is completed within 4-6 weeks to facilitate the personalized cancer vaccine to be used in time to stimulate the immune response of the subject with cancer.

In another preferred embodiment, the body fluid includes blood, urine, saliva, lymphatic fluid or semen.

In another preferred embodiment, the body fluid includes hydrothorax, ascites, or cerebrospinal fluid.

In another preferred embodiment, the method further includes step (h1): based on the DNA, RNA, and peptide chain synthesized in step (f), screening a single-chain antibody (scFV) that specifically binds to the secondarily selected sequence element and constructing and/or expanding a T cell (CAR-T) expressing chimeric antigen receptor (CAR), wherein the CAR contains the scFV as an extracellular antigen binding domain.

In another preferred embodiment, the single-chain antibody is obtained by single-chain antibody phage display technology.

In another preferred embodiment, in step (h1), for one or more (such as 2-5 kinds) of the secondarily selected sequence elements, screening respectively the specific single-chain antibodies (scFV), and constructing the corresponding T cells (CAR-T) expressing chimeric antigen receptor (CAR).

In another preferred embodiment, the T cell expressing chimeric antigen receptor (CAR-) is used for reinfusion to the subject.

In another preferred embodiment, the reinfusion further includes the additional administration of a CAR-T cell, TCR-T cell and/or co-stimulatory factor against a universal tumor antigen.

In another preferred embodiment, the method further includes step (h2): based on the DNA, RNA, and peptide chain synthesized in step (f), screening out a T cell receptor (TCR) that specifically binds to the secondarily selected sequence element, and constructing and/or expanding a T cell expressing the TCR (TCR-T).

In another preferred embodiment, in step (h2), for one or more (for example, 2-5 kinds) of the secondarily selected sequence elements, the specific TCR is screened out respectively, and corresponding T cell expressing the TCR is constructed and/or expanded.

In another preferred embodiment, the T cell expressing the TCR is used for reinfusion to the subject.

In another preferred embodiment, the reinfusion further includes the additional administration of a CAR-T cell, TCR-T cell and/or co-stimulatory factor against a universal tumor antigen.

In another preferred embodiment, the method further includes step (h3): based on the DNA, RNA, and peptide chain synthesized in step (f), the dendritic cell (DC) of the subject is subjected to priming treatment in vitro to obtain a primed dendritic cell.

In another preferred embodiment, in step (h3), multiple types (such as 2-5 or 5-10 or 10-20) of the secondarily selected sequence elements are used for priming treatment.

In another preferred embodiment, in step (h3), the method further comprises: co-cultivating the primed dendritic cell with the subject's T cell in vitro to prepare a DC-CTL cell.

In another preferred embodiment, the primed dendritic cell and/or DC-CTL cell are used for reinfusion to the subject.

In another preferred embodiment, steps (h1), (h2) and (h3) are independent and can be combined with each other arbitrarily.

In another preferred embodiment, in the method, step (g) is replaced with step (h1), (h2) and/or (h3).

In another preferred embodiment, steps (g), (h1), (h2) and (h3) are independent and can be combined with each other arbitrarily.

In another preferred embodiment, the normal fluid cell is selected from the group consisting of a peripheral blood mononuclear cell (PBMC).

In a second aspect of the present invention, it provides a personalized cancer vaccine, which is prepared by any of the methods of the first aspect of the present invention.

In another preferred embodiment, the vaccine further optionally contains an adjuvant.

In another preferred embodiment, the adjuvant includes: poly-ICLC, TLR, 1018ISS, aluminum salt, Amplivax, AS15, BCG, CP-870, 893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, remiquimod, SRL172, virus microbody and other virus-like particles, YF-17D, VEGF Trap, R848, β-glucan, Pam3Cys, Aquila QS21 stimulator, vadimezan or AsA404 (DMXAA).

In a third aspect of the present invention, it provides a cell product for immunotherapy, which is prepared by the method as described in the first aspect of the present invention, the cell product includes: a personalized CAR-T cell, personalized TCR-T cell, personalized primed DC cell and personalized DC-CTL cell.

In a fourth aspect of the present invention, it provides a method for inducing a tumor-specific immune response in a subject suffering from cancer, comprising administering to the subject in need the personalized cancer vaccine as described in the second aspect of the present invention.

In another preferred embodiment, the personalized cancer vaccine can also be used to prepare a pharmaceutical composition for combined administration of cancer treatment.

In another preferred embodiment, the personalized cancer vaccine and adjuvant can also be used in combination with other drugs and/or therapies.

In another preferred embodiment, the other drugs or therapies include anti-immunosuppressive drugs, chemotherapy, radiotherapy, or other targeted drugs.

In another preferred embodiment, the anti-immunosuppressive drugs include anti-CTLA-4 antibody, anti-PD1 antibody, anti-PD-L1 antibody, anti-CD25 antibody, anti-CD47 antibody or IDO inhibitor.

In another preferred embodiment, the pharmaceutical composition for treating cancer includes an antibody drug, cellular immunotherapy drug (such as a CAR-T cell, TCR-T cell, DC-CTL cell, etc.), and a combination thereof.

In a fifth aspect of the present invention, it provides a method for personalized treatment of a subject suffering from cancer, comprising administering to the subject in need the cell product of the immunotherapy as described in the third aspect of the present invention.

### DESCRIPTION OF FIGURE

FIG. 1 shows the observation of the body state of the mouse lung cancer animal model. Some mice in the experimental group (A, B, C) developed abdomen fur shedding after 4 weeks of injection, while the control group (D) performed normally.

FIG. 2 shows a single CTC schematic. CTC (as indicated by arrows) is isolated from the plasma of colon cancer patients (A) and cancer-bearing mice (B). The CTC enriched by Celsee system is stained and showed DAPI positive (blue), panCK positive (green) and CD45 negative. The recovery and enrichment of CTC cells are performed, and the final number of CTC cells sorted from the colon cancer patient is verified by Next Generation Sequencing (NGS) and analyzed by Sequenza software, the total number of cells is 10, of which CTC cell abundance (cellularity) accounts for 30-40%, and chromosome ploidy is mixed polyploid; background color indicates the possibility of analyzing log posterior probability (LPP) (blue=most likely, white=the least likely) (C).

FIG. 3 shows a schematic diagram of nucleic acid amplification of CTC single cell exome sequencing and transcriptome sequencing (G & T-seq). Isolating mRNA from plasma CTC enriched samples of cancer-bearing mice, reverse transcribing it into cDNA (A, B), and extracting the remaining genomic DNA and amplifying (C) for the use of exome sequencing and transcriptome sequencing.

FIG. 4 shows the sequencing results of a CTC mutation corresponding to the cDNA library of FIG. 3AB.

FIG. 5 shows a schematic diagram of the preparation of a personalized cancer vaccine in mice. 8-12 kinds of peptide vaccines have been screened and prepared, mixed with adjuvant and injected subcutaneously to the cancer-bearing mice to observe the efficacy. Cancer-bearing mice injected with personalized cancer vaccines are still alive, while cancer-bearing mice without vaccines dies one after another.

FIG. 6 shows a schematic diagram of the patient's ctDNA fragment size. Using the Agilent 2100 analyzer, the arrow above (provided by Rubicon) shows two fragments, on the left is the main 170 bp fragment, and on the right are some macromolecular fragments; the figure below shows a patient ctDNA sample, except for the main 170 bp fragment, the macromolecular fragments have been removed by proprietary enrichment methods.

FIG. 7 shows the predicted results of tumor neoantigen in cancer patients. HLAHD software is used to classify patients' HLA molecules, and Sentieon TNscope and other softwares are used to isolate the tumor neoantigen from the patient's CTC DNA exome sequencing sequences using the patient's peripheral blood mononuclear cell DNA exome sequencing sequences as controls. The correlation analysis software is used to predict the affinity of the short peptide chain tumor neoantigen and its corresponding wild-type short peptide chain to the patient's MHC molecule. The red box shows the best candidate components screened by the patient's personalized cancer vaccine. The affinity of the short peptide chain tumor neoantigen with MHC class I molecules (7.16 nM) is about 3550 times higher than that of its corresponding wild-type short peptide chain (25394.2 nM).

FIG. 8 shows a schematic diagram of cancer driver gene mutations in cancer patients. Exome sequencing results of two cancer patients (colon cancer and skin cancer) show that the Muc16 gene mutation has 5 identical sites (indicated by arrows).

FIG. 9 shows the tumor neoantigen screening process. Using proprietary screening methods, screening 80-100 tumor neoantigen candidates from cancer patient plasma CTC to form a tandem minigene (TMG) library for in vitro transcription (IVT), RNA molecules are transfected to DC cells isolated and differentiated from patient plasma; then the patient's peripheral blood is drawn, CD8 + T cells, CD4 + T helper cells are isolated, and ex vivo ELISPOT experiments are performed respectively, tumor neoantigen that can activate CD8 + T cells or CD4 + T helper cells can be screened out, and the personality cancer vaccine can be prepared.

FIG. 10 shows an experimental procedure for the preparation of CTC tumor neoantigen vaccine with noninvasive plasma and invasive hydrothorax and ascite separated from a ovarian cancer patient.

### DETAILED DESCRIPTION OF INVENTION

After extensive and intensive research, the present inventors has collected the body fluids for the first time to separate and enrich a certain percentage of circulating tumor cells (CTC) and their DNA and RNA or a mixture of circulating tumor DNA (ctDNA) and circulating tumor RNA (ctRNA) in the body fluids of subjects with cancer. Using next-generation sequencing technology (including sequencing methods such as ULP-WGS, WES, and RNA-seq), using DNA and RNA samples of other normal body fluid cells of subjects with cancer as control samples of CTC and its DNA and RNA, or using free DNA (cfDNA) and free RNA (cfRNA) samples from other normal cells in the body fluids of subjects with cancer as control samples of ctDNA and ctRNA, in the extracted and enriched CTC DNA and RNA and/or ctDNA and ctRNA fragments, 10-30 types of DNA, RNA or short peptide chains containing tumor-specific somatic cell mutations, that is tumor neoantigen that can cause protein sequence changes and can tightly bind to human HLA type I or II receptors and T-cell receptors (TCR), and can also activate CD8 + T cells or CD4 + T helper cells, which helps early diagnosis of cancer are isolated and confirmed. Moreover, personalized cancer vaccines can be prepared within 4-6 weeks, thereby developing rapid and efficient personalized solid tumor immunotherapy solutions. On this basis, the present invention has been completed.

Specifically, the present inventor has enriched (1) circulating tumor cells (CTC) and their DNA and RNA or (2) circulating tumor DNA (ctDNA) and circulating tumor RNA (ctRNA), and using sequencing technology (NGS) including Ultra low pass whole genome sequencing (ULP-WGS), whole exome sequencing (WES) and RNA-seq at a specific ratio, the DNA and RNA samples of other normal body fluid cells, which has accounted for ≤95% of the mixed extract of CTC from cancer patients and DNA and RNA from other normal body fluid cells, are used as the control samples of CTC and its DNA and RNA samples, which have accounted for ≥5%, or free DNA (cfDNA) and free RNA (cfRNA) samples from other normal cells that account for ≤95% of the body fluids of subjects with cancer are used as control samples for ctDNA and ctRNA samples that account for ≥5%, in the extracted and enriched CTC DNA and RNA and/or ctDNA and ctRNA fragments, 10-30 types of DNA, RNA or short peptide chains containing tumor-specific somatic cell mutations, that is tumor neoantigen that can cause protein sequence changes and can tightly bind to human HLA type I or II receptors and T-cell receptors (TCR), and can also activate CD8 + T cells or CD4 + T helper cells, which helps early diagnosis of cancer are isolated and confirmed. Moreover, personalized cancer vaccines can be prepared within 4-6 weeks, and promptly used to stimulate the immune response of subjects with cancer.

### Definition

“Body fluid (Bodily fluid)” refers to the fluid that is naturally present or secreted by the human body, including but not limited to blood, urine, saliva, lymph, semen, hydrothorax, ascites, cerebrospinal fluid, etc.

Circulating tumor cells (CTC) is a general term for various types of tumor cells that exist in the blood circulation system. Due to spontaneous or diagnostic operation, most of the CTCs undergo apoptosis or are engulfed after detaching from solid tumor lesions, including primary lesion and metastatic lesion and entering the peripheral blood due to the spontaneous or diagnostic treatments, and a few can escape and develop into metastases, increasing the risk of death of cancer patients.

“cfDNA and cfRNA” refer to the DNA and cellular RNA fragments from the patient's tumor genome that are constantly flowing in the human body fluid system, especially the blood circulation system. Normal cells and tumor cells will rupture. After the cells rupture, the DNA in the cells will be released into the body fluids. The DNA and RNA that enter the blood are called plasma free DNA (cfDNA) or cfRNA.

“ctDNA and ctRNA” refer to the DNA and cellular RNA fragments from the patient's tumor genome that are constantly flowing in the human body fluid system, especially the blood circulation system. The part of DNA and RNA derived from tumor cells in cfDNA and cfRNA above carries tumor-specific mutations, called ctDNA or ctRNA.

“Tumor neoantigen” refers to a new antigen that is only expressed on the surface of a certain tumor cell and does not exist on normal cells, so it is also called a unique tumor antigen. Such antigens can exist in tumors of the same tissue type in different individuals. For example, the melanoma-specific antigen encoded by the human malignant melanoma gene can exist in melanoma cells of different individuals, but normal melanocytes do not express them. Such antigens can also be shared by tumors of different histological types. For example, mutant ras oncogene products can be found in the digestive tract, lung cancer, etc. However, due to the difference of the amino acid sequence with normal proto-oncogene ras expression products, it can be recognized by the body's immune system, stimulating the body's immune system to attack and eliminate tumor cells. Tumor neoantigen mainly induces T cell immune response.

“WGS” is the one that based on obtaining certain genetic and physical map information, decomposing genomic DNA into small fragments of about 2 kb for random sequencing, supplemented by a certain number of 10 kb clones and BAC clone end sequencing, and using a supercomputer to integrate for sequence assembly.

“ULP-WGS” is an ultra-low-throughput, rapid and relatively inexpensive whole-genome sequencing method with a sequencing depth of only 0.01-0.1×, which has been applied to non-invasive prenatal screening to detect large-scale chromosomal abnormalities. It can be used for early screening of CTC and ctDNA in cancer patients. The screened positive CTC and ctDNA samples can be further analyzed by WES and RNA-seq.

“WES”: Exome refers to the sum of all exon regions in the genome of eukaryotes and contains the most direct information on protein synthesis. WES is a genomic analysis method for high-throughput sequencing after capturing and enriching the DNA of whole-genome exon region with known coordinates using a designed probe kit. For the human genome, the exon region accounts for about 1% of the genome, about 30M.

“RNA-seq”: Transcriptome refers to the sum of all RNA that can be transcribed in a cell or a group of cells under the same physiological conditions, including mRNA, rRNA, tRNA and non-coding RNA. RNA-seq is to extract the specific type of RNA to be studied, reverse transcribe it into cDNA, and use high-throughput sequencing technology to obtain almost all transcript sequence information of a specific tissue or organ of a species in a certain state.

“MHC” is a general term for all biocompatible complex antigens, which means that the molecules encoded by the MHC gene family (MHC class I, class II, class III) are located on the cell surface, and the main function is to bind peptide chains derived from pathogens and show pathogens on the surface of cells to facilitate T-cell recognition and perform a series of immune functions. MHC class I is located on the surface of general cells, and can provide some conditions within the general cell. For example, if the cell is infected by a virus, then the short peptide chains of the outer membrane fragments of the relevant virus are prompted outside the cell through the MHC, which can be used for identification by CD8 + T cells for killing. MHC class II is only located on antigen presenting cells (APC), such as macrophages, CD4 + T helper cells, etc. This kind of provision is the situation outside the cell. For example, if bacteria invade in the tissue, after macrophages have swallowed, the bacterial fragments are prompted to the helper T cells by MHC to start the immune response. MHC class III mainly encodes complement components, tumor necrosis factor (TNF), etc. Human MHC is usually called HLA (human leucocyte antigen), that is, human body fluid cell antigen. The MHC gene, located on the short arm of human chromosome 6, is highly polymorphic.

“CD8 + T cells” generally refers to T cells that express CD8 on the cell surface, and CD8 (cluster of differentiation 8) is a transmembrane glycoprotein, used as a co-receptor of TCR. Similar to TCR, CD8 is combined with MHC class I molecules for CD8 + T cell identification and killing.

“CD4 + T helper cells” usually refer to T helper cells that express CD4 on the cell surface, and belong to a body fluid cell, and CD4 (cluster of differentiation 4) is a glycoprotein, used as a co-receptor of TCR and assists TCR to recognize APC. CD4 is combined with MHC class II molecules for CD8 + T cell identification and killing.

“IC50” refers to the maximum half inhibitory concentration of the measured antagonist or inhibitor. It can indicate the half amount of a drug or substance (inhibitor) that inhibits certain biological procedures (or certain substances contained in this procedure, such as enzymes, cell receptors, or microorganisms).

“Immune adjuvant”, also known as non-specific immunoproliferative agent. It is not inherently antigenic, but injected into the body together with the antigen or in advance and can enhance immunogenicity or change the type of immune response.

The term “DNA, RNA, peptide chain” refers to DNA, RNA, and/or peptide chain.

“CAR-T”, the full name is chimeric antigen receptor T cell immunotherapy, is currently one of the more effective immunotherapy methods for malignant tumors. The chimeric antigen receptor (CAR) is the core component of CAR-T, giving T cells the ability to recognize tumor antigens in an HLA-independent manner, which allows CAR-modified T cells to recognize a wider range of targets than natural T cell surface receptor TCR. It has a good effect on the treatment of acute leukemia and non-Hodgkin's lymphoma. “TCR-T”, the full name is T cell receptor (TCR) chimeric T cells (TCR-T), is to improve the “affinity” of these TCRs to the corresponding tumor neoantigen by partial genetic modification to eliminate the tumor cell. The genetically modified TCR technology is also called the affinity-enhanced TCR technology. As two most recent immune cell technologies of the current adoptive cell reinfusion therapy ACT technology with the above-mentioned CAR-T, because they can express specific receptors and target specific cells such as tumor cells, they have received extensive attention and research.

“DC-CTL”, DC cells are impacted by autologous or tumor lysates of the same type, and can specifically present a certain type of tumor antigen, thereby inducing cytotoxic lymphocytes (CTL) targeting a specific tumor cell, which improves anti-tumor effect. A large number of clinical data at home and abroad show that DC-CTL immunotherapy combines all the advantages of DC and CTL, has obvious effects on many tumors, and has a positive effect on controlling the recurrence and metastasis of tumors, improving the immunity of patients, and improving the quality of life. DC-CTL has become one of the main treatment methods of current biological therapy, and also one of the most promising tumor treatment methods in the future to cure tumors.

CTC Enrichment and Extraction of CTC DNA and RNA

The type, number, and changes of CTCs have important clinical guidance significance in early cancer screening, tumor medication, efficacy evaluation, and relapse monitoring. But in the early tumor patients, 10 mL of blood contains only about 1-10 CTCs, so that it is difficult to collect rare CTCs in blood samples. At present, the principle of CTC enrichment mainly includes two methods: capturing based on cell size (filtration) and positive capturing based on tumor surface markers (immunology). Filtration method is not dependent on specific markers and can efficiently enrich or separate all types of CTCs, so that it is more widely used. Among the existing products that use filtration method to enrich CTCs, Celsee PREP100 and PREP400 systems are CTC products that do not require pre-removal of red blood cells, are highly automated, highly efficient enrichment, and integrate the cell enrichment system with the cell identification and analysis system (www.celsee.com). Cells do not need to be centrifuged, cell lysed, and do not add any labels; the sample demand is small; the sorting speed is fast; using microfluidic chip sorting technology, the sorting efficiency is as high as more than 80%; the automatic multi-channel setting, which can process 4 samples at a time. CTC can be subjected to in situ immunohistochemistry, DNA-FISH, RNA-FISH, cell culture, PCR and NGS analysis, etc. In addition, during the CTC enrichment process, its cell suspension inevitably contains other background body fluid cells such as leukocytes and lymphocytes, etc (Gogoi P et al. Methods Mol Biol 2017; 1634: 55-64). We here for the first time skillfully propose to use DNA and RNA samples of other background body fluid cells such as leukocytes and lymphocytes in the cell suspension as controls, and perform NGS analysis including ULP-WGS, WES, and RNA-seq on DNA and RNA of CTC to discover tumor-specific somatic cell mutations.

In a preferred embodiment, for a cell sample with a total of only 10 cells (wherein CTC is 1-4, that is, CTC accounts for 10-40%), the method of the present invention can still detect tumor-specific somatic cell mutations with high sensitivity.

Extraction and Enrichment of ctDNA and ctRNA

The size of ctDNA is about 166 bp, which is equivalent to the length around the ribosome and its linker. These DNA fragments derive from four parts: 1. Necrotic tumor cells; 2. Apoptotic tumor cells; 3. Circulating tumor cells; 4. Exosomes secreted by tumor cells. Humans have been studying ctDNA since it was discovered in 1977. In 1994, researchers for the first time identified DNA containing tumor-marking mutations derived from tumors. Coupled with the noninvasive and easy availability of ctDNA, the tumor markers found in it are considered to be used to detect early diagnosis, progression, and prognosis of tumors and personalized medication guidance. Although as early as 1987, Wieczorek et al. discovered that ctRNA was present in the plasma of cancer patients, it was not until 1999 that specific gene mRNA was continuously confirmed in the plasma of different cancer patients (Gonzalez-Masiá J A et al. OncoTargets & Therapy 2013; 6: 819-832). However, due to the extremely low content of ctDNA and ctRNA in human blood, only 1%, or even 1 in 10,000 of circulating DNA, there are great challenges in their detection. The inventors separated and removed cells from the body fluid samples of cancer patients, and extracted cfDNA and cfRNA from the cell-removed samples by molecular sieve, methylation separation, filter centrifugation, etc., and enriched ctDNA and ctRNA fragments up to 10-100%, which is beneficial to downstream WGS, WES and RNA-seq. In addition, during the enrichment of ctDNA and ctRNA, its nucleic acid suspension inevitably contains cfDNA and cfRNA from other normal cells in body fluids. The present invention here for the first time skillfully proposes to use cfDNA and cfRNA samples from other normal cells in body fluids in this nucleic acid suspension as controls, and perform NGS analysis including ULP-WGS, WES, and RNA-seq on ctDNA and ctRNA to find tumor-specific somatic cell mutations.

Isolation and Confirmation of Tumor Neoantigen

The main purpose of the present invention is to separate and enrich CTC and its DNA and RNA or ctDNA and ctRNA in the body fluids of cancer patients. Using NGS including ULP-WGS, WES and RNA-seq, DNA, RNA or short peptide chains containing tumor-specific somatic cell mutations, that is tumor neoantigen that can cause protein sequence changes and can tightly bind to human HLA type I or II receptors and TCR, and can also activate CD8 + T cells or CD4 + T helper cells are isolated and confirmed. It is especially important that these mutated neoantigen are only present in the patient's tumor cells, but not in the patient's normal tissues and cells, which is helpful for the early diagnosis of cancer. Significant mutations include: (1) non-synonymous mutations leading to changes in amino acid sequence; (2) read-through mutations leading to changes or disappearance of the stop codon, and forming a longer tumor-specific protein sequence at the C-terminus of the protein sequence; (3) mutations at the splice site leading to the appearance of tumor-specific protein sequences containing introns within the mRNA sequence; (4) chromosomal recombination resulting in the formation of a chimeric protein, wherein the binding site contains tumor-specific protein sequences (gene fusion); (5) frameshift mutation or deletion of mRNA resulting in a new protein open reading frame (ORF) containing tumor-specific protein sequences.

WES is a high-throughput sequencing of genomic DNA that is enriched directionally. It can sequence human exomes at relatively low cost. In 2009, the emergence of exome capture tools has made WES technology rapidly hot, and the current technology platform on the market is relatively mature. After WES isolates DNA, RNA or short peptide chains containing tumor-specific somatic cell mutations that can cause protein sequence changes, these mutations also require RNA-seq to confirm the expression of these DNA and RNA encoding the mutant proteins or variants. After extracting ctRNA from the aforementioned body fluid sample, removing rRNA, retaining the transcripts with and without PolyA, synthesizing the first strand of cDNA with random hexamers, and adding buffer, dNTPs, RNase H and DNA polymerase I to synthesize the second strand of cDNA, purifying it with PCR kit and eluting with EB buffer to repair the end, adding sequencing adapter, and performing PCR amplification to complete the entire library preparation work, and the constructed library is used for NGS.

In addition to using traditional WES and RNA-seq technologies to screen tumor neoantigens, modern novel bioinformatics can also be used to establish MHC (HLA type I or II receptor) binding libraries to screen peptide chains or RNA variants that can bind to MHC, narrowing the range of WES, especially RNA-seq, and accelerating the process of NGS experiments.

Tumor Neoantigens Binds to HLA Type I or II Receptors and TCR

There are various ex vitro prediction of HLA combined experimental methods in the art, such as the IEDB comprehensive prediction method, which can be used to predict the affinity of the isolated and confirmed potential tumor neoantigens to HLA, i.e., IC50≤100 nm or at least ≤150 nm. Based on the normal human body's lack of tolerance to the aforementioned completely novel protein sequence and their tumor specificity, as long as their predicted affinity with HLA type I or II receptors is ≤500 nM, they can be regarded as the short peptide chains that can be considered as the highest priority to make personalized vaccines. If the non-synonymous mutant short peptide chain has a predicted affinity of ≤150 nM for the HLA type I or II receptor and the corresponding natural peptide chain has a predicted affinity of ≥1000 nM for the HLA type I or II receptor, the short peptide chain can be used as a second priority to make a personalized vaccine. If the non-synonymous mutant short peptide chain and the corresponding natural peptide chain have a predicted affinity of ≤150 nM for the HLA type I or II receptor, the short peptide chain can be used as a third priority to make a personalized vaccine.

But combining with HLA alone is not an optimized immunogenicity prediction, and increasing the TCR binding degree can improve the prediction accuracy. However, the present invention proposes to extract T-cells from the body fluids of cancerous subjects, and use the screened short peptide chains or variants tcoding RNA for ex vitro TCR binding test and CD8 + T cell or CD4 + T helper cell activation test, this allows TCR to be integrated into traditional workflows, in order to better predict the accuracy of the new epitope bound to TCR.

Activation Test of Tumor Neoantigen CD8 + T Cell or CD4 + T Helper Cell Combined with HLA Type I or II Receptors and TCR

CD8 + T cells and CD4 + T helper cells isolated from cancerous subjects can be activated in vitro by co-cultivation with patient tumor neoantigen polypeptide chains that bind to HLA type I or II receptors and TCR, thereby secreting an IFN-γ (IFN-γ ELISPOT assay) against these tumor neoantigen polypeptide chains.

Preparing Personalized Cancer Vaccines for Cancer Patients

Adopting standard solid phase synthesis chemistry combined with reverse phase high performance liquid chromatography (RP-HPLC), DNA, RNA or short peptide chain personalized cancer vaccines containing tumor-specific somatic cell mutations, which can cause protein sequence changes, and can closely bind with human HLA type I or II receptors and TCR, and can also activate anti-tumor CD8 + T cells or CD4 + T helper cells, are prepared by GMP.

Speeding Up the Process of Personalized Cancer Vaccine Treatment

At present, the development and preparation of personalized cancer vaccines starts from the excision of cancerous tissues of patients, which takes about 6-8 weeks and is expensive, which is especially long for patients with metastatic cancer. The inventors have separated and enriched CTC and its DNA and RNA or circulating tumor DNA (ctDNA) and circulating tumor RNA (ctRNA) by collecting body fluids for the first time in the world. Using NGS including ULP-WGS, WES, and RNA-seq, using DNA and RNA samples of other normal body fluid cells of subjects with cancer as control samples of CTC and its DNA and RNA, or using free DNA (cfDNA) and free RNA (cfRNA) samples from other normal cells in the body fluids of subjects with cancer as control samples of ctDNA and ctRNA, in the extracted and enriched CTC DNA and RNA and/or ctDNA and ctRNA fragments, 10-30 types of DNA, RNA or short peptide chains containing tumor-specific somatic cell mutations, that is tumor neoantigen that can cause protein sequence changes and can tightly bind to human HLA type I or II receptors and T-cell receptors (TCR), and can also activate CD8 + T cells or

CD4 + T helper cells, are isolated and confirmed. The personalized cancer vaccines can be prepared within 4-6 weeks, providing a feasible reference for the development of rapid and efficient personalized solid tumors, especially metastatic cancer immunotherapy solutions, to partially meet the huge clinical treatment needs of cancer patients.

Use of Adjuvant

The immune adjuvant itself is not antigenic, but injection into the body together with the antigen or in advance can enhance immunogenicity or change the type of immune response. For example, in previous studies, Poly-ICLC showed an adjuvant function similar to the yellow fever vaccine, so that it is currently considered the best Toll-like receptor 3 agonist.

Cell Products for Immunotherapy

The present invention also provides cell products for personalized immunotherapy. Representative cell products include (but are not limited to): CAR-T cells, TCR-T cells, primed DC cells and DC-CTL cells.

In one embodiment, the method of the present invention includes: 2-5 single-chain antibodies (SCFV) having specificity and high affinity to the secondarily selected sequence elements (i.e. tumor neoantigen) are rapidly screened; the T cells in the peripheral blood of the subject (i.e., the subject with cancer) are collected, the CAR containing the scFV as the extracellular antigen binding domain is expressed in the T cell through in vitro recombinant DNA technology, thereby preparing a personalized CAR-T cell directed against the tumor neoantigen.

One or more (e.g., 2-5) personalized CAR-T cells of the present invention can be reinfused to the subject, thereby stimulating the cancerous subject to produce an immune response against solid cancer and/or blood cancer.

In one embodiment, the method of the present invention includes: rapidly screening 2-5 TCRs with specificity and high affinity to the secondarily selected sequence elements (i.e., tumor neoantigen) are rapidly screened; and then preparing T cells containing the corresponding TCR, that is, the personalized TCR-T cells against the tumor neoantigen.

One or more (e.g., 2-5) personalized TCR-T cells of the present invention can be reinfused to the subject, thereby stimulating the cancerous subject to produce an immune response against solid cancer and/or blood cancer.

In one embodiment, the method of the present invention includes: priming DC cells with multiple (e.g., 2-5, 5-10, or 10-20) secondarily selected sequence elements to obtain the primed DC cells. The corresponding DC-CTL cells are further prepared.

The primed dendritic cells and/or DC-CTL cells of the present invention can be reinfused to the subject, thereby stimulating the cancerous subject to produce an immune response against solid cancer and/or blood cancer.

Combination of Personalized Cancer Vaccine and Other Drugs and Therapies

Two groups of melanoma patients published online by Nature have relapsed after personalized cancer vaccine immunotherapy. For example, two stage IV patients (lung metastases) in the C Wu team still have cancer recurrence after receiving immunotherapy. However, after these patients received combination therapy with PD-1 antibody, the condition was under control. To a large extent, this should be related to changes in patients' immune pools after treatment with personalized cancer vaccines. Researchers from both teams in the US and Germany found that after specific vaccine treatment, most of the patients produced T cells that have specific binding ability to tumor neoantigen, and these T cells could not be detected in the blood before being immunized, that is, personalized cancer vaccines found those sleeping T cells from the patient's immune library, or induced by specific antigens to produce T cells that did not originally exist, and recruit them into the immune system to produce anti-cancer effects (Ott P A Nat 2017; 547: 217-221, Epub 2017 Jul. 5; Sahin U et al. Nat 2017; 547: 222-226, Epub 2017 Jul. 5). More importantly, most of these newly added T cells are PD-1 positive. It can be used in combination therapy with PD-1 antibody and other anti-immunosuppressive drugs including anti-CTLA-4 antibody, anti-PD-L1 antibody, anti-CD25 antibody, anti-CD47 antibody or IDO inhibitor. Therefore, personalized cancer vaccines against tumor neoantigen can expand the existing immune pool of patients through “immune recruitment”, “immunization induction” and other means, bringing new hope to cancer immunotherapy. At the same time, personalized cancer vaccines can also be used in combination with other drugs and therapies, including vaccine+chemotherapy, vaccine+radiotherapy, vaccine+other targeted drugs, etc.

Various references are cited in all parts of the invention. These references and their cited references are incorporated by reference into the present invention and disclosed in order to more fully describe the current status of work in the field of the present invention.

It should be understood that the above disclosure related to the preferred embodiments of the present invention and many variations does not depart from the scope of the present invention. The invention is further illustrated by the following examples, which cannot be interpreted in any way as limiting the scope of the invention.

The invention will be further illustrated with reference to the following specific examples. It is to be understood that these examples are only intended to illustrate the invention, but not to limit the scope of the invention. For the experimental methods in the following examples without particular conditions, they are performed under routine conditions (eg. Sambrook et al., Molecular Cloning: A Laboratory Manual (New York: Cold Spring Harbor Laboratory Press, 1989) or as instructed by the manufacturer. Unless otherwise specified, all percentages, ratios, proportions or parts are by weight.

Unless otherwise specified, the materials or reagents used in the examples are all commercially available products.

### Example 1. Establishment of a Mouse Early Lung Adenocarcinoma Model and Treatment with a Personalized Cancer Vaccine

It has been reported that subcutaneous injection of 1-methyl-3-nitro-1-nitroso-guanidine (MNNG, a strong cancer-inducing agent) in mice can induce the occurrence of lung cancer to establish an animal model of early lung cancer (Xiao S M et al. 2015; Acta Lab Anim Sci Sin 23: 227-32). We established a mouse model of early lung adenocarcinoma based on this method and treated it with a personalized cancer vaccine.

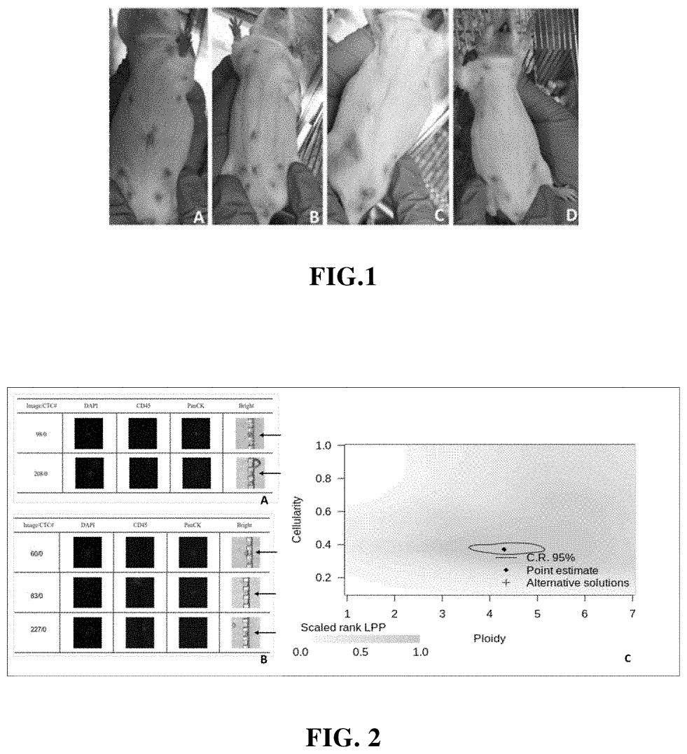

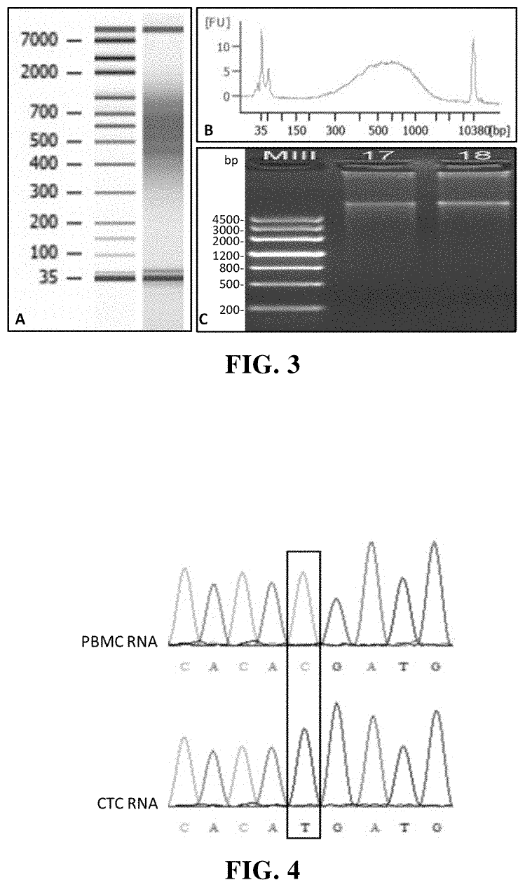

0.2 mL of nitrosoguanidine solution with a concentration of 2.0 mg/mL was injected subcutaneously into 20 KM female mice (25-30 g) every week for four consecutive weeks (FIG. 1). About 4 weeks after the injection, 200 ul of whole blood was taken from the tail of the mice to isolate and enrich CTC (FIG. 2A) and its DNA and RNA or ctDNA and ctRNA. FIG. 3 shows a schematic diagram of nucleic acid amplification of CTC single cell exome sequencing and transcriptome sequencing (G & T-seq). Isolating mRNA from plasma CTC enriched samples of cancer-bearing mice, reverse transcribing it into cDNA (FIG. 3, A, B), and extracting and amplifying the remaining genomic DNA (FIG. 3, C) for use in exome sequencing and transcriptome sequencing. FIG. 4 shows the sequencing results of a CTC mutation corresponding to the cDNA library of FIG. 3AB.

Using DNA samples from peripheral blood mononuclear cells of sick mice as controls, WES and RNA-seq were performed on the extracted and enriched CTC DNA and RNA fragments to isolate and confirm the short peptide chains that can cause protein sequence changes and contain tumor-specific somatic cell mutations. Since the coding gene of mouse MHC molecule is similar to that of human, using bioinformatics software, 8-12 short peptides containing tumor-specific somatic cell mutations, that is tumor neoantigen that can cause protein sequence changes and can tightly bind to MHC class I or II molecules and mouse TCR, and can also activate CD8 + T cells or CD4 + T helper cells are screened out.

The tumor neoantigen of mice were analyzed by the following methods: mouse H-2 typing software was used to classify mouse H-2 molecules, and using software such as Sentieon TNscope, the DNA exome sequencing sequence of the peripheral blood mononuclear cells of cancer-bearing mice was used as a control to isolate tumor neoantigen from the sequencing sequence of CTC DNA exome of cancer-bearing mice, and the correlation analysis software was used to predict the affinity of the short peptide chain tumor neoantigen and its corresponding wild-type short peptide chain to the mouse MHC molecule. A preferred tumor neoantigen peptide (KAIRNVLII) was screened from the personalized cancer vaccine of sick mice, and the affinity (9.19 nM) of the short peptide chain tumor neoantigen with MHC class I molecules is about 556 times higher than that of its corresponding wild-type short peptide chain (5105.43 nM); at the same time, IEDB predicts a higher affinity for mouse TCR (MHC I immunogenicity) score (0.20254).

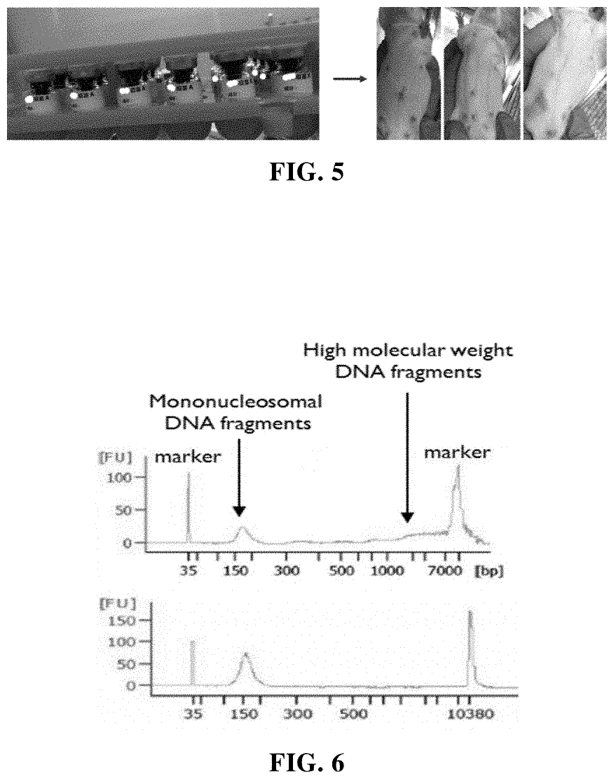

The above-mentioned preferred tumor neoantigen peptide was made into a personalized cancer vaccine, mixed with an adjuvant, and injected subcutaneously into cancer-bearing mice, and the efficacy was observed (FIG. 5). Cancer-bearing mice injected with personalized cancer vaccines were still alive, while cancer-bearing mice without vaccines died one after another.

### Example 2. Isolation and Enrichment of CTC and its DNA and ctDNA in Plasma of Cancer Patients, Using WES and RNA-Seq to Isolate and Confirm Tumor Neoantigen

Two tubes were collected from the peripheral blood of three cancer patients (lung cancer, colorectal cancer and bladder cancer), respectively, one tube of 10 ml and the other tube of 5 ml whole blood, placed in EDTA blood collection tube, and mixed up and down several times. The 10 ml tube was used for CTC enrichment and counting by the Celsee system (FIG. 2B).

During the CTC enrichment process, its cell suspension inevitably contains other blood cells such as leukocytes and lymphocytes. Here we used the DNA and RNA samples of other blood cells such as leukocytes and lymphocytes in this cell suspension as controls for the first time, and performed NGS analysis including ULP-WGS, WES, and RNA-seq on the DNA and RNA of CTC to discover tumor-specific somatic cell mutation. The final cell number of sorting was verified by NGS. For cell samples with a total of only 10 cells, CTC cell abundance (cellularity) accounts for 30-40%, and chromosomal ploidy is a mixed polyploid; the background color indicates the possibility of analyzing the log posterior probability (LPP) (blue=most likely, white=least likely) (FIG. 2C).

For another 5 ml of whole blood, the blood sample was centrifuged for 10 minutes at 1900 ×g (3000 rpm) and 4° C. The supernatant was removed carefully without disturbing the lower suction, about 3 ml of plasma can be obtained from a 5 ml whole blood sample. The supernatant was transferred to two 1.5 ml EP tubes and centrifuged at 16000×g and 4° C. for 10 minutes. The supernatant was carefully removed without disturbing the small amount of precipitate formed by high-speed centrifugation, and stored in a −80° C. refrigerator. After day 2, 3 ml plasma samples were taken to extract cfDNA with QIAamp free nucleic acid extraction kit (Qiagen 55114), centrifugation and filtration steps were added, and ctDNA was enriched. At the same time, Rubicon's ThruPLEX Plasma-seq kit was used to amplify ctDNA with less content before NGS analysis (FIG. 6).

In addition, during the ctDNA enrichment process, its nucleic acid suspension inevitably contains cfDNA from other normal cells in body fluids. Here we for the first time used the cfDNA samples from other normal cells in body fluids in this nucleic acid suspension as a control. ctDNA was analyzed using NGS including ULP-WGS, WES, and RNA-seq to discover tumor-specific somatic cell mutations.

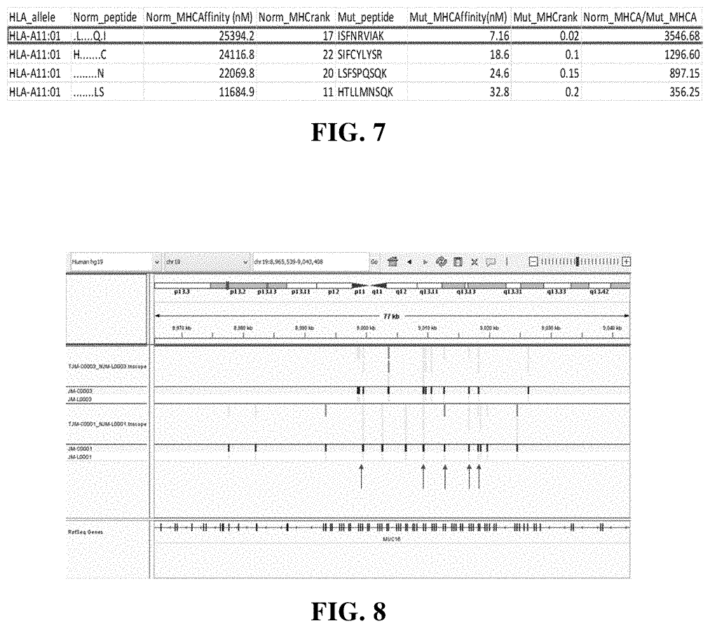

The above samples were directly subjected to nucleic acid extraction, amplification, and then second-generation sequencing including exome sequencing. Analysis was performed using Sentieon's related software processes including TNscope, etc. Based on comparing tumor exome and transcriptome data with normal cell control data, simultaneous detection of mutant peptides produced by multiple mutations, and combined with advanced neoantigen prediction algorithms and software, high-quality tumor neoantigen short peptide sequences were quickly and efficiently screened out (FIG. 7).

The exome sequencing results of two cancer patients (colon cancer and skin cancer) show that the cancer driver gene Muc16 gene mutation has 5 identical sites (FIG. 8).

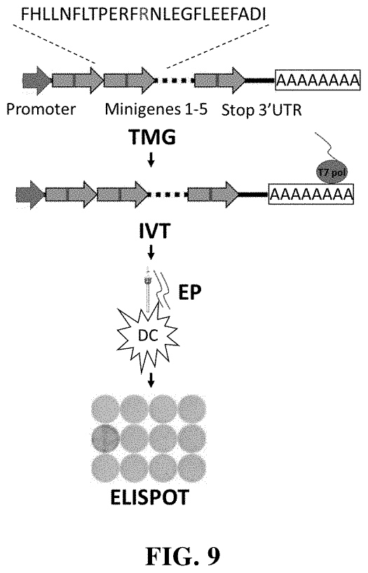

Screening 80-100 tumor neoantigen candidate components from cancer patient plasma CTC, forming a tandem minigene (TMG) library, performing in vitro transcription (IVT), and transfecting RNA molecules into DC cells isolated and differentiated from patient plasma; then drawing the patient's periphery blood, isolating CD8+T cells and CD4+T helper cells, and ex vivo ELISPOT experiments were performed to screen out tumor neoantigen antigens that can activate CD8+T cells or CD4+T helper cells to make personalized cancer vaccine (FIG. 9).

Using conventional affinity-based methods to screen tumor neoantigen, the hit rate is only 3%, while using the HLA-agnostic method of the present invention to screen tumor neoantigen, the hit rate can be increased to 35%.

### Example 3. Separation of Non-Invasive Plasma and Invasive Hydrothorax and Ascite in Patients with Ovarian Cancer to Prepare CTC Tumor Neoantigen Vaccine

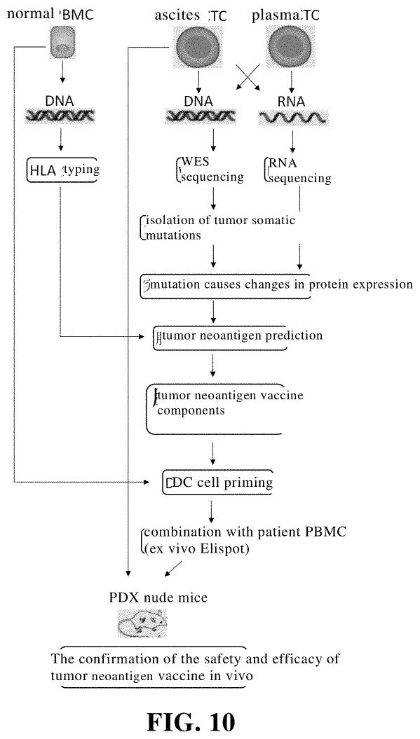

The main content of this embodiment is to carry out the following preclinical animal experiments (see experimental procedure in FIG. 10):

1. Separating and enriching CTC. CTC was isolated and enriched from noninvasive plasma (10 ml) and invasive ascites in patients with advanced ovarian cancer with ascites.

2. In vitro culture of ascites CTC to establish a PTX model of nude mice with CTC ascites in patients with ovarian cancer.

3. Extraction and next-generation sequencing of plasma and ascites CTC RNA and DNA. Using Next Generation Sequencing Technology (NGS) including Whole Exon Sequencing (WES) and RNAseq to isolate and confirm 10-30 types of short peptide chain containing tumor specific somatic cell mutation, that is, tumor neoantigen which can cause protein sequence changes and can be closely combined with patient MHC molecules and TCR, and also can activate CD8+T cells or CD4+T helper cells from the plasma and ascites CTC.

4. The effectiveness of tumor neonatal antigen vaccine was confirmed in vivo. Tumor neoantigen peptide or mRNA vaccine screened from ascites and plasma CTC after second-generation sequencing and subsequent bioinformatics analysis was ex vivo combined with DC isolated from the patient's plasma (priming), and verified by ex vivo Elispot experiment to screen an appropriate number of tumor neoantigen vaccine components, which was combined with PBMC isolated from the patient's plasma, and injected together into the tail vein of PDX nude mice.

5. The status of some humanized PDX mice was observed daily, and the size of subcutaneous tumors of PDX mice was measured every two days to evaluate the safety and effectiveness of personalized cancer vaccines and further explore the pharmacodynamic characteristics.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A method for preparing a personalized cancer vaccine, comprising the following steps:
(a) providing a first sample sequencing data set A1 and a first control sequencing data set R1 corresponding to a subject; and/or providing a second sample sequencing data set A2 and a second control sequencing data set R2 corresponding to a subject,
wherein the first sample data set A1 and the first control sequencing data set R1 are obtained by a method including the following steps:
t1) providing a first sample, and the first sample is a sample containing a CTC cell and a normal body fluid cell;
t2) performing CTC cell enrichment treatment on the first sample, thereby obtaining an enriched first sample, wherein in the enriched first sample, the CTC cell abundance C1≥5% and the normal body fluid cell abundance C2≤95%, based on the total number of all cells in the enriched sample, and the ratio of the CTC cell abundance C1 to the normal body fluid cell abundance C2 is recorded as B1 (i.e., B1=C1/C2);
t3) extracting DNA and/or RNA from the enriched first sample, thereby obtaining a first nucleic acid sample, wherein the first nucleic acid sample includes a nucleic acid sample from a CTC cell and a nucleic acid from a normal body fluid cell; and
t4) sequencing the first nucleic acid sample, wherein the nucleic acid sample from a normal body fluid cell in the first nucleic acid sample is used as a control for the nucleic acid sample from a CTC cell, thereby obtaining the first sample sequencing data set A1 and the first control sequencing data set R1, wherein the first sample sequencing data set A1 corresponds to the sequencing data set of a CTC cell, and the first control sequencing data set R1 corresponds to the sequencing data set of a normal body fluid cell;
wherein the second sample data set A2 and the second control sequencing data set R2 are obtained by a method including the following steps:
w1) providing a second sample, and the second sample is a sample containing a circulating tumor DNA (ctDNA) and a circulating tumor RNA (ctRNA) and other free DNA (cfDNA) and free RNA (cfRNA);
w2). enriching the second sample to obtain an enriched second nucleic acid sample; wherein, the enriched second nucleic acid sample includes ctDNA and ctRNA from a CTC cell and cfDNA and cfRNA from a normal body fluid cell, wherein based on the total weight of all nucleic acids, the content of ctDNA and ctRNA L1≥5%, while the content of cfDNA and cfRNA from a normal cell L2≤95%, and the ratio of the content L1 to L2 is recorded as B2 (i.e., B2=L1/L2);
w3). sequencing the second nucleic acid sample, wherein the cfDNA and cfRNA from a normal cell in the second nucleic acid sample are used as a control for ctDNA and ctRNA from a CTC cell to obtain a second sample sequencing data set A2 and a second control sequencing data set R2, wherein the second sample sequencing data set A2 corresponds to the sequencing data set of a CTC cell, and the second control sequencing data set R2 corresponds to the sequencing data set of a normal body fluid cell;
(b). performing sequence alignment treatment on the first sample sequencing data set A1 and the first control sequencing data set R1, or the second sample sequencing data set A2 and the second control sequencing data set R2, respectively, thereby obtaining a first candidate data set S1 or a second candidate data set S2; wherein any sequence element in the first candidate data set S1 is an element present in the A1 but not present in the R1; and any sequence element in the second candidate data set S2 is an element present in the A2 but not present in the R2;
(c). performing an HLA type I or II receptor affinity prediction analysis on any sequence element in the first candidate data set S1 and/or the second candidate data set S2 to obtain a primarily selected sequence element, the primarily selected sequence element is a sequence element that binds tightly to the HLA type I or II receptor (IC50≤500 nm, preferably, 100 nm);
(d). based on the primarily selected sequence element, synthesizing a DNA, RNA, short peptide chain corresponding to the primarily selected sequence element;
(e). using the synthesized DNA, RNA, and short peptide chain to perform an in vitro T-cell receptor (TCR) binding test and CD8 + T cell and/or CD4 + T helper cell activation test to obtain 10-30 secondarily selected sequence elements, wherein the secondly selected sequence elements can bind to TCR and activate CD8 + T cells and/or CD4 + T helper cells;
(f) based on the secondarily selected sequence elements, synthesizing DNA, RNA and peptide chains corresponding to the secondarily selected sequence elements;
(g). mixing the DNA, RNA, and peptide chains synthesized in the previous step with a pharmaceutically acceptable carrier to prepare a pharmaceutical composition, which is a personalized cancer vaccine.

2. The method of claim 1, wherein in the classification and/or analysis, for the two types of sequencing data D1 and D2 at the same location or position, if the following Formula Q1 is met, the sequencing data D1 is classified as CTC sequencing data, and the sequencing data D2 is classified as sequencing data of a normal body fluid cell
RD1/(RD1+RD2)/(C1+C2) (Q1)
wherein,
RD1 is the frequency of occurrence (or abundance, such as read depth) of sequencing data D1 (such as read or a related sequence thereof)
RD2 is the frequency of occurrence (or abundance, such as read depth) of sequencing data D2 (such as read or a related sequence thereof)
C1 is the abundance of a CTC cell in the enriched first sample;
C2 is the abundance of a normal body fluid cell in the enriched first sample.

3. The method of claim 1, wherein in the classification and/or analysis, for the two types of sequencing data E1 and E2 at the same location or position, if the following Formula Q2 is met, the sequencing data E1 is classified as ctDNA and ctRNA sequencing data of a CTC cell, and the sequencing data E2 is classified as ctDNA and ctRNA sequencing data of a normal cell
RE1/(RE1+RE2)≈L1/(L1+L2) (Q2)
wherein
RE1 is the frequency of occurrence (or abundance, such as read depth) of sequencing data E1 (such as read or a related sequence thereof)
RE2 is the frequency of occurrence (or abundance, such as read depth) of sequencing data E2 (such as read or a related sequence thereof)
L1 is the content of ctDNA and ctRNA of a CTC cell in the enriched second sample;
L2 is the content of ctDNA and ctRNA of a normal cell in the enriched second sample.

4. The method of claim 1, wherein the sequence element is the following group:
a DNA sequence element, RNA sequence element, and/or peptide chain sequence element.

5. The method of claim 4, wherein the DNA sequence element contains 2-5 DNA variants, and each DNA variant contains at least 5 short peptide chain coding sequences; and/or
the RNA sequence element contains 2-5 RNA variants, and each RNA variant contains at least 5 short peptide chain coding sequences; and/or
the peptide chain sequence element contains 5-100 amino acids.

6. The method of claim 1, wherein the normal body fluid cell includes leukocyte, monocyte, lymphocyte and the like.

7. The method of claim 1, wherein the body fluid includes blood, urine, saliva, lymphatic fluid or semen.

8. The method of claim 1, wherein the method further includes step (h1): based on the DNA, RNA, and peptide chain synthesized in step (f), screening a single-chain antibody (scFV) that specifically binds to the secondarily selected sequence element and constructing and/or expanding a T cell (CAR-T) expressing chimeric antigen receptor (CAR), wherein the CAR contains the scFV as an extracellular antigen binding domain.

9. The method of claim 1, wherein the method further includes step (h2): based on the DNA, RNA, and peptide chain synthesized in step (f), screening out a T cell receptor (TCR) that specifically binds to the secondarily selected sequence element, and constructing and/or expanding a T cell expressing the TCR (TCR-T).

10. The method of claim 1, wherein the method further includes step (h3): based on the DNA, RNA, and peptide chain synthesized in step (f), the dendritic cell (DC) of the subject is subjected to priming treatment in vitro to obtain a primed dendritic cell.

11. The method of claim 10, wherein in step (h3), the method further comprises:
co-cultivating the primed dendritic cell with the subject's T cell in vitro to prepare a DC-CTL cell.

12. A personalized cancer vaccine, which is prepared by the methods of claim 1.

13. The vaccine of claim 12, wherein the vaccine further optionally contains an adjuvant.

14. The vaccine of claim 13, wherein the adjuvant includes: poly-ICLC, TLR, 1018ISS, aluminum salt, Amplivax, AS15, BCG, CP-870, 893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, remiquimod, SRL172, virus microbody and other virus-like particles, YF-17D, VEGF Trap, R848, β-glucan, Pam3Cys, Aquila QS21 stimulator, vadimezan or AsA404 (DMXAA).

15. A personalized CAR-T cell, wherein the personalized CAR-T cell is prepared by the method of claim 8.
